(19) United States
(12) Patent Application Publication
(10) Pub. No.: US 2013/0130243 A1
(43) Pub. Date: May 23, 2013

(54) METHOD AND DEVICE FOR DETECTING AND QUANTIFYING AN ANALYTE WITH RECYCLING OF THE REAGENTS

(71) Applicants: Thierry Livache, Jarrie (FR); Roberto Calemczuk, Grenoble (FR); Malika Amdaoud, Grenoble (FR)

(72) Inventors: Thierry Livache, Jarrie (FR); Roberto Calemczuk, Grenoble (FR); Malika Amdaoud, Grenoble (FR)

(73) Assignee: Commissariat a L'Energie Atomique ET Aux Energies Alternatives, Paris (FR)

(21) Appl. No.: 13/643,991

(22) PCT Filed: Apr. 22, 2011
(86) PCT No.: PCT/EP2011/056502

§ 371 Date: Feb. 8, 2013

(30) Foreign Application Priority Data
Apr. 28, 2010 (FR) 1053274

(51) Int. Cl. G01N 33/543

(52) U.S. Cl. CPC G01N 33/543; G01N 33/54366
USPC 435/6.11; 435/7.4; 435/7.2; 435/7.1; 436/501; 435/7.92; 435/6.1; 435/287.2; 435/287.1; 422/69

(57) ABSTRACT

The present invention relates to a method for detecting and quantifying an analyte present in a liquid of interest using a solid support, the surface of which comprises at least one active area on which at least one probe capable of binding said analyte is immobilized and a solution containing at least one secondary reagent capable of binding to the analyte, said method comprising a step consisting of recycling said solution in order to put it back into contact with the surface and notably with the active area at least one additional time. The present invention also relates to a device which may be applied within the scope of such a method.

### TECHNICAL FIELD

The present invention refers to the field of analytical devices and methods, notably used for detecting and/or quantifying an analyte in a liquid of interest. Such devices are also known under the name of [symbol] biosensors [symbol] or [symbol] biochips [symbol] .

More particularly, within the scope of biological analyses conducted with biorecognition methods on a support, the present invention proposes a method and a device which allow recycling of the secondary reagents applied and not having been adsorbed on the surface of the sensor so as to reuse them after their passing over the latter.

### STATE OF THE PRIOR ART

Functionalized supports are known and have been used for several decades for identifying analytes of interest such as nucleotide sequences, antibodies or molecules of various chemical nature and notably proteins.

Generally, a biofunctionalized support has, grafted on its surface, specific probes having affinities for analytes in solution such as antibodies or antigens, directed against a well-defined target analyte or molecule. This support is adapted to a detection system with which adsorption of the target analyte or molecule may be shown on the surface of the sensor. This detection may apply detectable secondary reagents such as for example marked antibodies recognizing an antigen of the analyte or of the target molecule.

The surface of the support is maintained under a flow of a solution such as an experimental medium or a notably aqueous sample to be analyzed. To do this, a fluidic device is placed above an active area of the support: it consists of one (or more) inlet route(s), of an interaction chamber, and of one (or more) outlet route(s). Upstream from the inlet route, an injection system is optionally found, with which a well-defined volume of the sample to be analyzed may be injected (FIG. 1A).

Certain analyses may require the injection of one (or more) secondary reagent(s) allowing adsorption of the analyte at the surface of the support, to be expressed as an experimentally exploitable signal. Such secondary reagents may be fluorescent markers [1].

To do this, a secondary reagent having good affinity for the analyte to be detected is injected onto the support. These secondary reagents thus bind to the molecules of analytes present at the surface of the support. This chemical secondary reagent should moreover also have properties making it easily detectable by the selected readout system. Thus it is quite possible to resort to several secondary reagents, by using chemical entities having successive chemical affinities with each other. These techniques are often designated as [symbol] sandwich [symbol] techniques.

The analysis of a sample may take place in several steps [2].

The support is first maintained under a flow of an experimental medium before a sample of given volume potentially containing the analyte to be detected is injected onto the support via a given injection system. When the totality of the sample to be analyzed has circulated over the support, the return into the experimental medium is accomplished automatically. Alternatively, the sample may also be put into continuous circulation on the support.

Next, by a (similar) method, the user injects a solution containing the secondary reagent. In the case of [symbol] multiple [symbol] development requiring several secondary reagents, the user injects the secondary reagents in succession.

This microfluidic device is an [symbol] open [symbol] system i.e. the chemical entities which have not adhered to the support are discharged, via the outlet route, towards a biological garbage bin. More specifically, the technique as described earlier requires systematic consumption of secondary reagents for each studied sample and this whether the analyzed sample is negative (i.e. it does not contain any target molecule) or positive. From the moment that the probability of analyzing a contaminated sample is low, this system has the drawback of generating unnecessary expense in terms of secondary reagents.

Moreover, with such a device, the analysis of the sample requires two successive injections: that of the sample possibly containing the analyte, and then the injection of the secondary reagent. Thus, for each sample to be analyzed, a new batch of secondary reagents has to be injected.

Recycling of reagents is a method conventionally used in chemistry, like during distillation with reflux. For complex synthesis, certain reagents may be recycled, such as for example in the case of the synthesis of hydrazines described in patent U.S. Pat. No. 6,605,265 [3]. International application WO 98/49187 describes the use of recycling within the scope of the synthesis of complex bio-organic molecules which are peptoid oligomers [4].

On the other hand, in the field of biological analysis, it is common to use single-use reagents. Only the reactive area may be recycled in the case of biosensors (cf. FIG. 1).

Therefore, a real need exists for a device and a method in the field of biological analysis with which the consumption of secondary reagents may be limited without affecting the detected signal or even by improving it.

### DISCUSSION OF THE INVENTION

The present invention proposes a device and a method using such a device with which it is possible to overcome the drawbacks of the methods of the state of the art as discussed earlier.

Most particularly, the present invention relates to a method for detecting and optionally quantifying an analyte possibly present in a liquid of interest. The method according to the invention comprises the following steps:

i) putting said liquid of interest into contact with the surface of a solid support comprising at least one active area on which at least one probe capable of binding said analyte is immobilized;

ii) putting said surface into contact with a solution containing at least one either directly or indirectly detectable secondary reagent, capable of either directly or indirectly binding to the analyte;

iii) detecting and optionally quantifying said secondary reagent immobilized on said active area, either directly or indirectly;

the method according to the present invention further comprising a step consisting of recycling said solution in order to put it again into contact with the surface at least once.

Within the scope of the method according to the present invention and of its different embodiments or alternatives, the steps for contact with the secondary reagent and for detection and optional quantification may be successive or simultaneous.

The method according to the present invention is distinguished from the method of the state of the art by applying the recycling step and therefore repeatedly putting the solution containing the secondary reagent into contact with the surface of the support. However, the notion of recycling does not exclusively include such repeated contacting. It also includes the fact that the solution containing a secondary reagent may be recovered and then used for applying another method according to the invention either with a same support but with another liquid of interest, or with a distinct support.

In the method according to the invention, the number of times during which the solution containing the secondary reagent is put into contact with the surface of the support and notably with the active area(s) (step (ii)+recycling step(s)) is an integer comprised between 2 and 1,000, notably between 2 and 200 and in particular between 2 and 20. One skilled in the art will be able to determine, without any inventive effort, the most suitable number of contacting operations depending on different parameters such as the stability of the secondary reagent.

In a 1^{st} alternative of the method according to the invention, the recycling step is applied just after the first contact of the surface of the support with the secondary reagent.

In this alternative, the method according to the invention therefore comprises the following steps:

a) putting said liquid of interest in contact with the surface of a solid support comprising at least one active area on which at least one probe capable of binding said analyte is immobilized;

b) putting said surface in contact with a solution containing at least one either directly or indirectly detectable secondary reagent, capable of binding to the analyte either directly or indirectly;

c) recycling said solution so as to repeat at least once step (b) and optionally step (c);

d) detecting and optionally quantifying, said immobilized secondary reagent on said active area, either directly or indirectly.

Because of this step, the secondary reagent having not been bound with the analyte immobilized on the active area is recycled so that it may be put again into contact with the surface of the support and notably with the active area(s) which it has. If putting the secondary reagent in contact with the surface of the support is accomplished more than twice, repetition of step (c) is not an option.

In a 2^{nd} alternative of the method according to the invention, the recycling step is applied before putting the surface of the support in contact with a liquid of interest (i.e. prior to step (i)).

In this case, the method according to the invention comprises the following steps:

a₁) putting a 1^{st} liquid into contact with the surface of a solid support comprising at least one active area on which at least one probe capable of binding to said analyte is immobilized;

b₁) putting said surface into contact with at least one either directly or indirectly detectable secondary reagent, capable of binding to the analyte, either directly or indirectly;

c₁) recycling said secondary reagent which has not been immobilized on said active area by direct or indirect binding with the analyte;

d₁) detecting and optionally quantifying said secondary reagent immobilized on said active area, either directly or indirectly,;

a₂) putting said liquid of interest in contact with said surface;

b₂) putting said surface in contact with at least the secondary reagent recycled during said step (c₁);

d₂) detecting and optionally quantifying, said secondary reagent immobilized on said active area, either directly or indirectly.

Within the scope of this 2^{nd} alternative, the 1^{st} liquid applied may be a [symbol] control [symbol] liquid which is recognized as not containing the analyte to be detected. Consequently, the detection and optional quantification during step (d₁) allows detection and optionally quantification of the background noise of the experiment. The 1^{st} so-called [symbol] control [symbol] liquid may be a liquid of the same nature as the tested liquid of interest. As an example, mention may be made of the case when the analyte to be detected is an anti-measles antibody, the [symbol] control [symbol] liquid is a serum from a subject who has never contracted measles and for whom the anti-measles antibodies of maternal origin have completed disappeared.

Within the scope of this 2^{nd} alternative, the 1^{st} applied liquid may also be a liquid of interest according to the present invention, which, once step (d₁) is carried out, was recognized as not containing the sought analyte.

Within the scope of this 2^{nd} alternative, the 1^{st} applied liquid may also be a control liquid containing known amounts of the analyte(s) thereby used for calibrating the system.

A 3^{rd} alternative of the method according to the invention combines the two previous alternative. The different embodiments envisioned for these previous alternatives also apply to this third alternative. The latter comprises the following steps:

a₁′) putting a 1^{st} liquid into contact with the surface of a solid support comprising at least one active area on which at least one probe capable of binding said analyte is immobilized;

b₁′) putting said surface into contact with at least one either directly or indirectly detectable secondary reagent, capable of binding to the analyte either directly or indirectly;

c₁′) recycling said secondary reagent having not been immobilized on the active area by direct or indirect binding with the analyte;

d₁′) detecting and optionally quantifying said secondary reagent immobilized on the active area, either directly or indirectly;

a₂′) putting said liquid of interest into contact with said surface;

b₂′) putting said surface into contact with at least the secondary reagent recycled during said step c₁′);

c₂′) recycling said secondary reagent having not been immobilized on the active area by direct or indirect binding with the analyte so as to repeat at least once step (b₂′) and optionally step (c₂′);

d₂′) detecting and optionally quantifying said secondary reagent immobilized on the active area, either directly or indirectly.

The liquid of interest applied within the scope of the present invention is a liquid which may contain the analyte to be detected and optionally to be quantified. It may be of very diverse nature and origin.

This liquid of interest is advantageously selected from the group consisting of a biological fluid; a plant fluid such as sap, nectar and root exudates; a sample in a culture medium or in a biological culture reactor such as a cell culture of higher eukaryotes, yeasts, fungi or algae; a liquid obtained from one or more animal or plant cells; a liquid obtained from an animal or plant tissue; a sample in a food matrix; a sample in a chemical reactor; tap water, river water, sea water, water from air-cooled towers; an air sample, a sample from a liquid or gas industrial effluent; an earth sample or one of their mixtures.

A liquid of interest should not be understood as a solution prepared by the experimenter in which a compound which may be considered as an analyte has possibly been introduced in a known amount. Thus, a solution containing a secondary reagent as defined hereafter is not a liquid of interest according to the invention.

The biological fluid is advantageously selected from the group consisting of blood, such as full blood or anti-coagulated full blood, blood serum, blood plasma, lymph, saliva, spittle, tears, sweat, sperm, urine, stools, milk, cerebrospinal fluid, interstitial liquid, a fluid isolated from bone marrow, mucus or a fluid from the respiratory, intestinal or genito-urinary tract, cell extracts, tissue extracts and organic extracts. Thus, the biological fluid may be any fluid naturally secreted or excreted from a human or animal body or any fluid recovered from a human or animal body, by any technique known to one skilled in the art such as extraction, sample-taking or washing. The steps for recovering and isolating these different fluids from the human or animal body are carried out prior to applying the method according to the invention.

Also, if one of the contemplated samplings does not allow application of the method of the invention, for example because of its gas or solid nature, of its concentration or of the elements which it contains such as solid residues, waste, suspension or interfering molecules, the method of the invention further comprises a preliminary step for preparing the liquid of interest with optionally putting the sample into solution with techniques known to one skilled in the art such as filtration, precipitation, dilution, distillation, mixing, concentration, lyses, etc.

The analyte to be detected and optionally to be quantified in the liquid of interest may be selected from the group consisting of a molecule of biological interest; a molecule of pharmacological interest; a toxin; a carbohydrate; a peptide; an antigen; an epitope; a protein; a glycoprotein; an enzyme; an enzymatic substrate; a nuclear or membrane receptor; an agonist or antagonist of a nuclear or membrane receptor; a hormone; a polyclonal or monoclonal antibody; an antibody fragment such as a Fab, F(ab′)₂. Fv fragment or a hyper-variable domain or CDR for “Complementarity Determining Region”; a nucleotide molecule; an eukaryotic cell; a prokaryotic cell and a virus.

The expression [symbol] nucleotide molecule [symbol] used herein is equivalent to the following terms and expressions: [symbol] nucleic acid [symbol] , [symbol] polynucleotide [symbol] , [symbol] nucleotide sequence [symbol] , [symbol] polynucleotide sequence [symbol] . By [symbol] nucleotide molecule [symbol] , is meant within the scope of the present invention, a chromosome; a gene; a regulating polynucleotide; an either single strand or dual strand DNA, genomic, chromosomal, chloroplastic, plasmid, mitochondrial, recombinant or complementary DNA; total RNA; messenger RNA; ribosomal RNA (or ribozyme); transfer RNA; a sequence acting as an aptamer; a portion or a fragment thereof.

The probe used for functionalizing the active area of the solid support applied within the scope of the present invention is any molecule capable of forming a binding pair with the analyte to be detected, the probe and the analyte corresponding to the two partners of this binding pair. The bonds applied in the analyte-probe bond are advantageously non-covalent bonds and of low energy such as hydrogen bonds or Van der Waals bonds.

The probe used is therefore dependent on the analyte to be detected. Depending on this analyte, one skilled in the art will be capable without any inventive effort, of selecting the most suitable probe. It may be selected from the group consisting of a carbohydrate; a peptide; an antigen; an epitope; a protein; a glycoprotein; an enzyme; an enzymatic substrate; a membrane or nuclear receptor; an agonist or antagonist of a membrane or nuclear receptor; a toxin; a polyclonal or monoclonal antibody; an antibody fragment such as a Fab, F(ab′)₂, Fv or a hyper-variable domain (or CDR for “Complementarity Determining Region”); a nucleotide molecule as defined earlier; a peptide nucleic acid and an aptamer such as a DNA aptamer or an RNA aptamer.

The solid support of the device according to the invention may be any support allowing application of this invention. This may for example be a biochip support such as those conventionally used, in silicon, in glass, in metal, in polymer or in plastic. It may be of diverse size and shape.

The surface of the solid support has one or more active areas, these active areas may either be organized randomly or not. An active area corresponds to a pad, a spot (or a dot) or a chemically defined surface portion.

The surface of this support and notably the active area may advantageously consist of a conducting material if electrical or electrochemical functionalization is required. It may consist of any other material which may be used for grafting the probe if other functionalization techniques are selected, for example chemical functionalization techniques. It may be in a chemically or biologically modified material so that the probe may be attached thereon. It may be the actual surface of the support or of a coating deposited on this support by customary deposition techniques known to one skilled in the art, allowing functionalization by the probe. This coating may for example be silicon; glass; silicon dioxide allowing silanization; a suitable conducting (co)polymer such as those used for making biochips, in particular for attaching molecule probes of biochips such as polypyrrole; a metal such as gold, silver, platinum, for example in order to achieve electro-grafting, for forming self-assembled mono-layers, etc. The adhesion area may be delimited for example by the localization of the probes which functionalize it.

Generally, the functionalization of the active area by the probe which consists in an immobilization by the probe on the active area may be achieved by means of customary chemical or electrochemical grafting techniques ( [symbol] electro grafting [symbol] ), for example such as those described in documents [5] and [6].

The secondary reagent, applied and recycled within the scope of the method according to the present invention, is any molecule capable of binding to the analyte.

This binding may be direct. In this case, the secondary reagent and the analyte are capable of forming a binding pair, the secondary reagent and the analyte corresponding to both partners of this binding pair. The bonds applied in the analyte-secondary reagent bond are advantageously non-covalent bonds and of low energy as defined earlier for the analyte-probe bond.

When the analyte-secondary reagent bond is direct, the latter may be selected from the group consisting of a carbohydrate, a peptide; an antigen; an epitope; a protein; a glycoprotein; an enzyme; an enzymatic substrate; a membrane or nuclear receptor; an agonist or an antagonist of a membrane or nuclear receptor; a hormone; a polyclonal or monoclonal antibody; an antibody fragment such as a Fab, F(ab′)₂, Fv fragment or a hyper variable domain or CDR for [symbol] Complementarity Determining Region [symbol] ; a nucleotide molecule such as defined earlier; a peptide nucleic acid; a molecular beacon and an aptamer such as a DNA aptamer or an RNA aptamer.

By [symbol] molecular beacon [symbol] is meant a nucleotide molecule appearing as hairpin with a deactivated fluorophore, the fluorescence of the fluorophore being restored when the beacon binds to the analyte appearing as a complementary nucleotide sequence.

One skilled in the art will be able to determine, without any inventive effort, the composition of the solution in which the secondary reagent(s) is(are) contained. This composition will mainly depend on the nature of the secondary reagent(s) and on the nature of the analyte-probe bonds and analyte-secondary reagent bonds.

The bond between the secondary reagent applied and recycled within the scope of the method according to the invention and the analyte may be an indirect bond. In this case, the analyte-secondary reagent bond may involve at least one 3^{rd} partner also designated as a [symbol] 2^{nd} secondary reagent [symbol] , the latter being capable of directly binding, to the analyte on the one hand and to the 1^{st} secondary reagent on the other hand. Alternatively, it is possible to envision the use of several 2^{nd} secondary reagents among which at least one directly binds the analyte, at least one other directly binds the 1^{st} secondary reagent and at least one other binds two 2^{nd} secondary reagents.

Such an indirect bond may apply, when the analyte to be detected is a protein, a 2^{nd} secondary reagent which is a specific antibody for said protein, marked with biotin and a 1^{st} recycled secondary reagent which is a detectable streptavidin.

Such an indirect bond may also apply, when the analyte to be detected is a protein, a 2^{nd} secondary reagent which is a specific primary antibody for said protein and a 1^{st} recycled secondary reagent which is a marked secondary antibody directed against a species-specific portion of the primary antibody.

Within the scope of an indirect bond, the method according to the present invention may not only comprise a step for recycling the 1^{st} secondary reagent but also a step for recycling the 2^{nd} secondary reagent(s).

In the methods of the present invention, the secondary reagent applied and recycled within the scope of the method according to the present invention is either directly or indirectly detectable.

This direct or indirect detection and this optional quantification of the secondary reagent may apply techniques without any marker such as techniques using surface plasmon resonance (SPR for Surface Plasmon Resonance) or quartz scales.

Alternatively, direct or indirect detection and optional quantification may apply a marker.

The secondary reagent applied and recycled is directly detectable when it bears such a marker. It is indirectly detectable when a 3^{rd} secondary reagent capable of either directly or indirectly binding to this 1^{st} secondary reagent bears such a marker.

Regardless of whether detection is direct or indirect, the marker which may be used within the scope of the present invention is notably selected from the group consisting of:

a colored particle such as a colored latex particle which may produce, when it is aggregated on the active area, a signal visible to the naked eye;

a fluorophore or fluorescent marker such as fluorescein, rhodamine, phycobiliprotein, a [symbol] quantum dot [symbol] and Alexa fluorophores;

a phosphorescent marker such as a metal complex of one or several metals such as ruthenium, osmium, platinum, copper, molybdenum and chromium;

a chemiluminescent marker such as luminol or dioxetane;

a chemical molecule such as digoxigenin;

an electrochemically active molecule such as ferrocene;

a biologically active molecule capable of producing a detectable signal when it is incubated with the adequate enzyme or chromogenic substrate. This biologically active molecule is for example an enzyme such as alkaline phosphatase, horseradish peroxidase, luciferase or a protease or a substrate such as para-nitrophenyl phosphate, diaminobenzidine, luciferin; and

a radioactive marker such as an isotope notably of phosphorus [³²P], sulfur [³⁵S], hydrogen [³H] or iodine [¹²⁵I].

In the methods according to the present invention, the expression [symbol] put into contact with [symbol] is equivalent to the expression [symbol] circulate over [symbol] . The solid support surface is put into contact with the 1^{st} liquid, a liquid of interest or the secondary reagent as defined earlier. In the present invention the entire surface of the solid support may be put into contact or only a portion of the latter, provided that the involved portion comprises at least one active area as defined earlier. It is possible to refer to an [symbol] active surface [symbol] of the support, the latter corresponding to or encompassing the whole of the active areas present at the surface of the solid support.

In the methods according to the present invention, one (or more) step(s) for rinsing the surface of the support, notably after steps (a), (a₁), (a₂), (a₁′) and/or (a₂′) and/or prior to steps (d), (d₁), (d₂), (d₁′) and/or (d₂′), may also be carried out. The rinsing solution is preferably a solution which preserves the probe-analyte bonds and the direct or indirect analyte-secondary reagent bonds, such as a phosphate buffer or an aqueous solution.

Also, prior to the detection steps (d), (d₁), (d₂), (d₁′) and/or (d₂′), the surface of the support and notably the active area(s) may be dried and covered with a suitable medium for facilitating this detection.

The detection during steps (d), (d₁), (d₁′), (d₂) and (d₂′) applies a technique adapted to the marker used in the methods of the invention. This technique may be a technique allowing measurement of radioactivity, absorbence, fluorescence, the angle of refraction of light, the modulation of the wavelength, a potential difference, a change in resonance frequency or a change in reflectivity.

During steps (d), (d₂) and (d₂′), the presence of a signal at the active area on which one or more probe(s) has (have) been immobilized beforehand is an indication of the presence, in the liquid of interest applied, of a given analyte capable of binding to this probe.

Further, once the method has been calibrated, for example by measuring the signal obtained for variable amounts of analyte (standard curves), the signal obtained for a particular liquid of interest may be an indication of the amount or of the concentration of the analyte in this liquid. In this case, the method and the device according to the invention may be used not only for detecting but also for quantifying a given analyte in a liquid of interest.

The present invention also relates to a device which may be applied within the scope of a method as defined earlier.

The device according to the invention comprises:

a chamber (1) comprising a solid support, the surface of which comprises at least one active area which may be functionalized by at least one probe capable of binding an analyte;

a 1^{st} fluidic system adapted for circulating a liquid of interest which may contain said analyte over said surface;

a 2^{nd} fluidic system adapted for circulating a solution comprising at least one secondary reagent at least twice over said surface.

The chamber (1) comprising a solid support, the surface of which comprises at least one active area which may be functionalized by at least one probe capable of binding an analyte, is a structure conventionally present in biosensors or biochips of the state of the art.

The device of the present invention may for example comprise, for marketing purposes, a solid support including a non-functionalized surface. The user of this device may then easily, by means of standard techniques for functionalizing a surface of biochips, functionalize this surface in order to generate thereon at least one active area with one (or more) probe(s) which is (are) selected depending on the analyte which he/she desires to retain, in order to obtain a device allowing application of one of the methods of the invention.

The device of the present invention may also appear, for marketing purposes, as already including functionalized active area(s) by one (or more) probe(s) capable of binding to a particular analyte in order to attach it. This then gives the possibility of applying one of the methods of the invention immediately, without any prior functionalization of the surface of the solid support, for the analyte matching said probe.

The device according to the present invention comprises a biological garbage bin, reservoirs (or chambers for introducing or circulating fluid) in order to contain the liquid of interest and the solution(s) comprising the secondary reagents and suitable elements for circulating the liquid of interest and the solution(s) from these reservoirs and for bringing them onto the surface of the solid support and notably on the active surface of the support. It is clear that the elements suitable for circulating the liquid of interest or the solutions from reservoirs or compartments containing them, as far as the (active) surface of the solid support (or from the surface to the reservoirs), are different from said reservoirs and compartments.

Such elements are notably selected from the group consisting of conduits (or channels), connectors, one (or more) fluidic loop(s), pumps, peristaltic pumps, syringe pumps, valves notably injection valves, flow control devices and robinets (or gates) and any other system allowing displacement of fluids, in particular integrated microfluidic systems [7].

For secondary reagents, the device according to the present invention may have different reservoirs each containing one or more different secondary reagents.

In a particular embodiment, the 2^{nd} fluidic system of the device according to the present invention is adapted so as to circulate at least one solution comprising at least one secondary reagent, at least twice in the same direction over the same surface.

In this embodiment, the 2^{nd} fluidic system comprises at least one reservoir (7) containing a solution comprising at least one secondary reagent.

In fact, the present invention relates to a device which may be applied within the scope of a method as defined earlier, comprising:

a chamber (1) comprising a solid support, the surface of which comprises at least one active area which may be functionalized by at least one probe capable of binding an analyte;

a 1^{st} fluidic system adapted so as to circulate a liquid of interest which may contain said analyte over said surface;

a 2^{nd} fluidic system comprising at least one reservoir (7) containing a solution comprising at least one secondary reagent, said fluidic system being suitable for circulating a solution comprising at least one secondary reagent at least twice in the same direction over said surface.

It is clear that at each circulation of the solution comprising at least one secondary reagent over the surface, the solution is caused to pass beforehand into the reservoir (7), at least momentarily. The fluidic system therefore forms a fluidic loop comprising the reservoir (7).

Thus, the 2^{nd} fluidic system may comprise a single reservoir (7) containing a solution comprising at least one secondary reagent.

Alternatively, the 2^{nd} fluidic system may comprise several reservoirs (7) each containing an advantageously different solution comprising at least one secondary reagent.

In another particular embodiment, the 2^{nd} fluidic system of the device according to the present invention is suitable for circulating at least one solution comprising at least one secondary reagent at least once in one direction and at least once more in the opposite direction over said surface of the support and notably over the active surface.

In this embodiment, the 2^{nd} fluidic system comprises one (or more) upstream reservoir(s) (7 or 7a) containing the solution(s) comprising at least one secondary reagent, and optionally one or more reservoirs (7b) positioned downstream containing or which may contain the solution(s) comprising at least one secondary reagent.

In this embodiment, the 2^{nd} fluidic system may comprise one (or more) upstream reservoir(s) (7) containing the solution(s) comprising at least one secondary reagent, and another reservoir (7b) positioned downstream.

Alternatively, the 2^{nd} fluidic system may comprise an upstream reservoir (7a) containing the solution comprising at least one secondary reagent and a reservoir (7b) positioned downstream.

In another alternative, the 2^{nd} fluidic system may comprise several upstream reservoirs (7a) containing the solutions comprising at least one secondary reagent and several reservoirs (7b) positioned downstream. Advantageously, the number of upstream and downstream reservoirs is identical.

The device according to the present invention may also comprise means suitable for allowing detection and optional quantification of a secondary reagent immobilized on the active area. These means are adapted to detection and optional quantification without any marking or marker. Alternatively, these means are adapted to the marker which makes the secondary reagent detectable either directly or indirectly. The markers contemplated within the scope of the present invention are commonly used, one skilled in the art will be able to determine without any inventive effort, the elements to be used for detecting and optionally quantifying the signal emitted by the marker.

Other features and advantages of the present invention will further become apparent to one skilled in the art upon reading the examples below given as an illustration and not as a limitation, and referring to the appended figures.

### SHORT DESCRIPTION OF THE DRAWINGS

FIG. 1 shows a device using a method for the biorecognition on a support and for detect on of the signal according to the state of the art. FIG. 1A shows this system maintained under flow. FIG. 1B shows this system subject to the injection of an amplifying chemical entity, following the injection of the sample containing the analyte to be detected, the excess reagents (sample and amplifying chemical entity) being discharged towards the biological garbage bin. The numerical references of FIG. 1 correspond to the elements bearing the same references in FIG. 2, explained hereafter and have the same functions as these elements.

FIG. 2 proposes different alternatives of the devices according to the present invention and allowing recycling of at least one secondary reagent. FIGS. 2A and 2B show a device of the [symbol] flow reversal [symbol] type. FIG. 2C shows a loop recycling device.

FIG. 3 proposes a schematic illustration of a device according to the invention with two injection valves allowing recycling. FIG. 3A proposes a 1^{st} state of the device with the 1^{st} injection valve allowing injection of the liquid of interest into the main fluidic circuit and the 2^{nd} valve insulating the secondary circuit containing the secondary reagents of the active area. FIG. 3B proposes a 2^{nd} state of the device in which the secondary reagents are injected onto the active area which is isolated from the main fluidic circuit through the respective action of the 2^{nd} and of the 1^{st} valves.

FIG. 4 is a schematic illustration of the assembly with which SPR imaging may be achieved.

FIG. 5 is a schematic illustration of the method for amplification with functionalized gold nanoparticles.

FIG. 6 shows the time-dependent variations of reflectivity. The experiments shown in FIGS. 6A and 6B include 3 steps: (1) injection of the sample to be analyzed, followed by (2) injection of secondary antibodies, and then (3) circulation of gold nanoparticles over the active area. Both vertical lines delimit the duration during which the closed circuit containing the gold nanoparticles is in contact with the active area. FIG. 6A: the analyzed sample does not contain any target molecules. FIG. 6B: the analyzed sample contains target molecules.

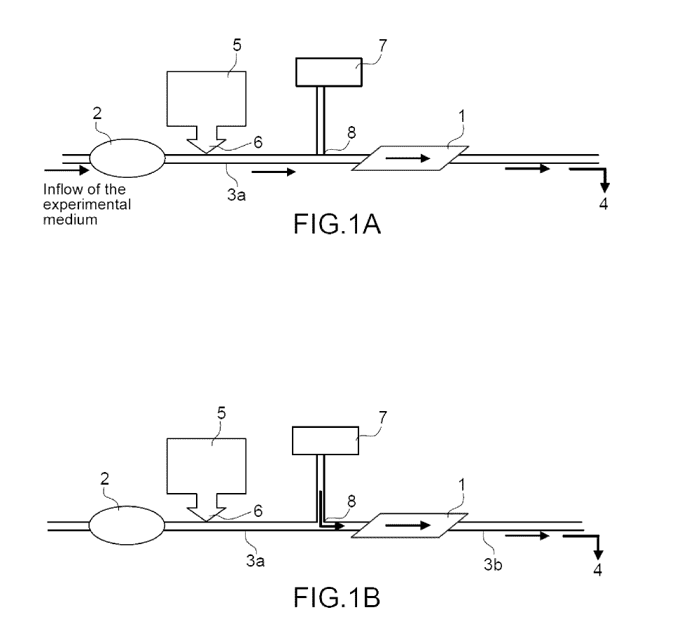

### DETAILED DISCUSSION OF PARTICULAR EMBODIMENTS

I. Devices According to the Present Invention:

I.1. Devices According to the Present Invention Using Flow Inversion.

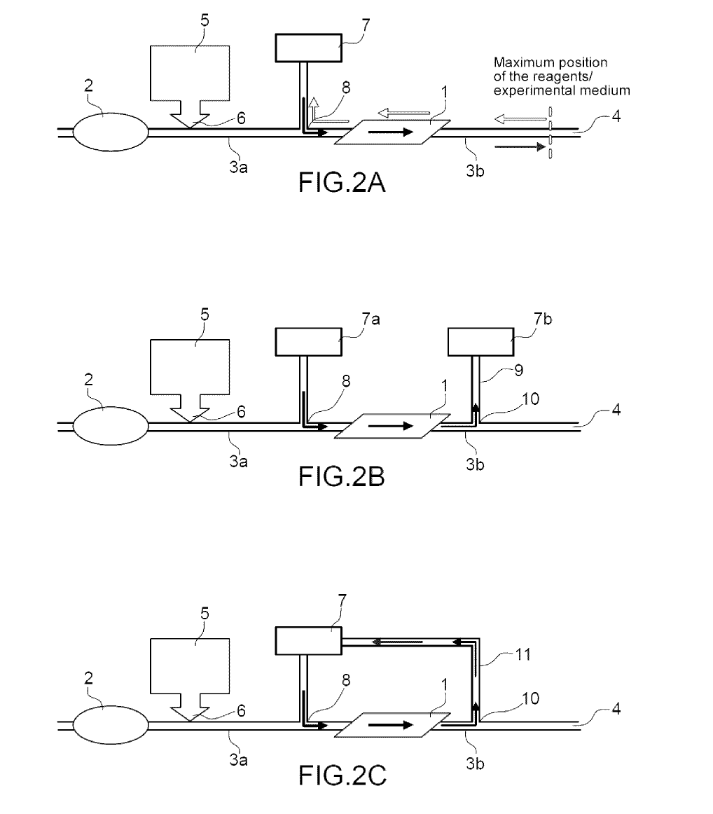

i. The Device Illustrated in FIG. 2A

FIG. 2A shows a device of the [symbol] flow reversal [symbol] type. With this device it is possible to inject the solution containing the secondary reagent onto the support in a direction and then in the opposite direction in order to reload the reservoir. In this configuration, recycling consists in directly reinjecting the reagents onto the active area.

This device comprises a chamber (1) in which the support with the active area(s) adapted for immobilizing the sought analyte is found as well as a main fluidic system fluidically connected to this chamber. This main fluidic system comprises a fluidic conduit (3a) connected to the chamber (1) on the one hand and to a pump (2) on the other hand, and a fluidic conduit (3b) connected to the chamber (1) on the one hand and to a biological garbage bin (4) on the other hand. The fluidic conduits (3a) and (3b) are advantageously in plastic material notably thermostable plastic such as polyetheretherketone (PEEK), polyvinyl chloride (PVC), polypropylene (PP), polyethylene (PE) polysiloxane (PDMS) or a material based on Teflon materials.

With the main fluidic system, it is possible, by means of the pump (2), to maintain a fluidic carrier current maintaining the chamber (1) and therefore the active surface and the active area(s) of the support under flow. The fluidic carrier current also designated as [symbol] experimental medium [symbol] is a fluid which neither reacts with the fluid of interest or the [symbol] control [symbol] fluid, nor with the secondary reagent(s). This may be a phosphate buffer.

The device also comprises a module for introducing the liquid of interest, i.e. a suitable system for introducing (or injecting) the liquid of interest (5) into the chamber (1). This system has a reservoir comprising the liquid of interest, of the syringe type, connected to the fluidic conduit (3a) at an introduction point (6). This may for example be a 6-way injection valve (of the HPLC type) provided with an injection loop containing the liquid of interest. Once it is introduced into the fluidic carrier current, the liquid of interest circulates from the fluidic conduit (3a) towards the chamber (1) and then towards the biological garbage bin (4) via the fluidic conduit (3b). The fluidic carrier current added with the liquid of interest is removed via the biological garbage bin (4).

The device further comprises a module suitable for introducing the solution containing at least one secondary reagent and for recycling this solution. This system comprises a reservoir (7) comprising the solution containing at least one secondary reagent, connected to the fluidic conduit (3a) at an introduction point (8). This may for example be an injection valve, a connector or a 3-way solenoid valve, one connected to the fluidic conduit (3a), the other one to the conduit (3b) and the latter to the conduit leading to the reservoir (7). The introduction point (8) is located between the point for introducing the liquid of interest (6) and the chamber (1). Alternatively, the introduction point for the liquid of interest (6) may be located between the introduction point (8) and the chamber (1). Once it is introduced to the fluidic carrier current, the solution containing the secondary reagent circulates from the fluidic conduit (3a) towards the chamber (1) and then towards the biological garbage bin (4) via the fluidic conduit (3b) and this, by means of a pump placed between the reservoir (7) and the introduction point (8) or downstream from the chamber (1); or a syringe pump, the syringe in this case acting as a reservoir (7); or programmable multi-channeled pumps; or a pneumatic system operating by pressurization-depressurization; these systems may be integrated into a microfluidic system.

However, before the fluidic carrier current added with the solution containing the secondary reagent reaches the biological garbage bin (4), the current is reversed by reversing the flow performed by the pump. The reversed current passes from the fluidic conduit (3b) to the chamber (1) and then to the fluidic conduit (3a) and to the reservoir (7).

An alternative of the device shown in FIG. 2A is a device having several reservoirs (7) each comprising a solution containing one (or more distinct) secondary reagent(s).

ii. The Device Illustrated in FIG. 2B.

The device of FIG. 2B differs from the one of FIG. 2A by the module suitable for introducing the solution containing at least one secondary reagent and for recycling this solution. The other elements of the device of FIG. 2B are identical with the elements of FIG. 2A bearing the same numerical references and have the same function as the latter.

The module adapted for introducing the solution containing at least one secondary reagent and for recycling this solution, of the device of FIG. 2B comprises a 1^{st} reservoir (7a) comprising the solution containing at least one secondary reagent, connected to the fluidic conduit (3a) at an introduction point (8) of the injection valve type, of the connector type or of the 3-way solenoid valve type. The introduction point (8) is located between the point for introducing the liquid of interest (6) and the chamber (1).

Alternatively, the point for introducing the liquid of interest (6) may be located between the introduction point (8) and the chamber (1). Once it is introduced into the fluidic carrier current, the solution containing the secondary reagent circulates from the fluidic conduit (3a) towards the chamber (1) and then towards the biological garbage bin (4) via the fluidic conduit (3b) and this, by means of a pump placed between the reservoir (7a) and the introduction point (8) or between the reservoir (7b) and the introduction point (10); or by a syringe pump, the syringe in this case acting as a reservoir (7a) or (7b) or by programmable multi-channel pumps; or by a pneumatic system operating by pressurization-depressurization; these systems may be integrated into a microfluidic system.

An alternative of the device shown in FIG. 2B is a device having several reservoirs (7a) each comprising a solution containing one (or more distinct) secondary reagent(s) and several reservoirs (7b) intended to each recover and recycle a particular solution.

However, before the fluidic carrier current added with the solution containing the secondary reagent reaches the biological garbage bin (4), it is deflected towards an ancillary route distinct from the fluidic conduit (3b) leading to the garbage bin (4). The deflection (10) may be accomplished by means of the same injection valve as the one optionally used at the injection point (8). It is also possible to use a valve with 3 inlet ways. This ancillary way is a fluidic conduit (9) fluidically connected to a 2^{nd} reservoir (7b) which may contain the solution containing at least one secondary reagent. In this case, circulation of the solution containing at least one secondary reagent over the active area in the chamber (1) may alternately be accomplished in one direction (1^{st} reservoir (7a) chamber (1)→2^{nd} reservoir (7b)) and then in the other direction (2^{nd} reservoir (7b)→chamber (1)→1^{st} reservoir (7a)).

Moreover, successive reversals of the direction of the flow, both in the device of FIG. 2A and in the one of FIG. 2B, may be repeated a large number of times thereby allowing an increase in the secondary reagents/active area interaction time.

I.2. Devices According to the Present Invention Without Flow Reversal.

i. The Device Illustrated in FIG. 2C.

The device of FIG. 2C differs from that of FIG. 2B by a portion of the module suitable for introducing the solution, containing at least one secondary reagent, and for recycling this solution. The other elements of the device of FIG. 2C are identical with the elements of FIGS. 2A and 2B bearing the same numerical references and have the same function as the latter.

After having circulated over the active area, the solution containing the secondary reagent circulates in an ancillary route to the one leading to the biological garbage bin. The ancillary route then ends up at the reservoir with the initial sample. In this case, the sample may continuously circulate over the active area.

Thus, the device of FIG. 2C comprises a single reservoir (7) comprising the solution containing at least one secondary reagent, connected to the fluidic conduit (3a) at an introduction point of the injection valve type, of the connector type or of the 3-way solenoid valve (8) type. The introduction point (8) is located between the point for introducing the liquid of interest (6) and the chamber (1). Alternatively, the point for introducing the liquid of interest (6) may be located between the introduction point (8) and the chamber (1). Once it is introduced into the fluidic carrier current, the solution containing the secondary reagent circulates from the fluidic conduit (3a) to the chamber (1) and then towards the biological garbage bin (4) via the fluidic conduit (3b) and this, by means of a recycling pump of the peristaltic or other type, optionally integrated to the fluidic system, connected to the reservoir (7) either upstream, or downstream from the chamber (1).

However, before the fluidic carrier current added with the solution containing the secondary reagent reaches the biological garbage bin (4), it is deflected towards an ancillary route distinct from the fluidic conduit (3b) leading to the garbage bin (4). The deflection (10) may be accomplished by means of the same injection valve as the one optionally used at the injection point (8). It is also possible to use a valve with 3 inlet ways. This ancillary way is a fluidic conduit (11) fluidically connected to the reservoir (7) comprising the solution containing at least one secondary reagent. In this case, the circulation of the solution containing at least one secondary reagent over the active area in the chamber (1) may be accomplished continuously in the same direction (reservoir (7)→conduit (3a)→chamber (1)→conduit (3b)→conduit (11)→reservoir (7)).

In this case, the circuit (reservoir of solution containing a secondary reagent (7) and chamber (1)) forms a loop and the circulation of the reagent over the active area is ensured as long as the flow is maintained (for example via a peristaltic pump).

It is obvious that suitably placed valves allow recirculation of the excess reagent. Thus, this device has the advantage of not imposing a time limit on the secondary reagent/active area interaction. The optional excess reagent may theoretically be circulated over the active area for an illimited time.

An alternative of the device shown in FIG. 2C is a device having several reservoirs (7) each comprising a solution containing one (or more distinct) secondary reagent(s).

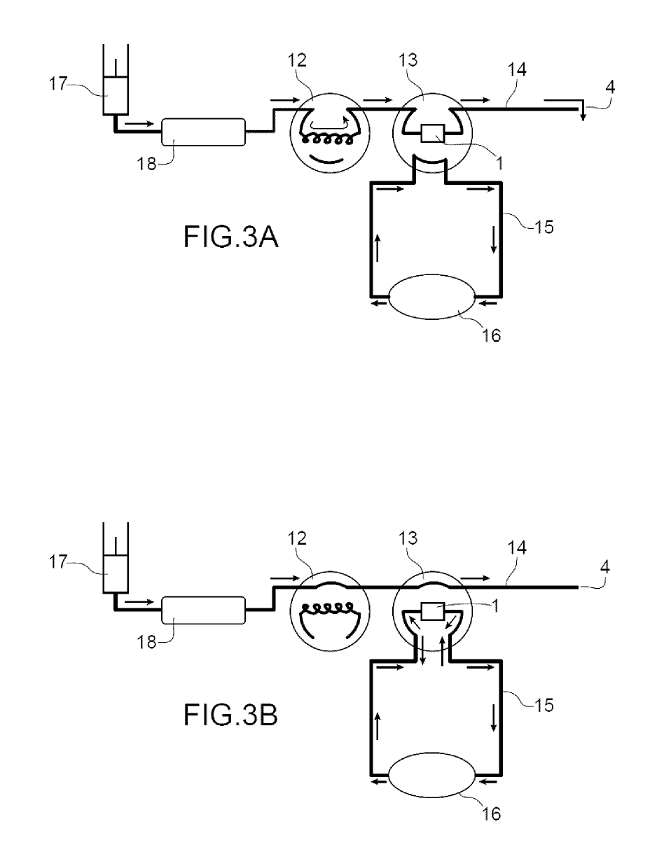

ii. The Device of FIG. 3.

FIG. 3 proposes a schematic illustration of a device illustrating the method and the device described in FIG. 2C.

FIG. 3A proposes the device in which the sample to be analyzed is injected from a reservoir (17) by means of a first injection valve (12) into the chamber (1) and then towards the biological garbage bin (4) and this, via the main fluidic circuit (14). With a second injection valve (13), it is possible to isolate the main fluidic circuit (14) which may have a degasser (18) capable of degassing the liquid of interest, from a secondary circuit (15) containing the solution with the secondary reagents.

This secondary circuit (15) is maintained under flow via a peristaltic pump (16).

FIG. 3B proposes the same device in which the reagent has already been injected into the chamber (1) and therefore at the active area. The first injection valve (12) has been switched so as to isolate the chamber (1) from the main fluidic circuit (14). The second valve (13) is actuated by hand. It gives the possibility of putting the active area in the chamber (1) in contact with the secondary circuit (15) containing the reagents, while isolating it from the main fluidic circuit (14). The solution containing the secondary reagents circulates in the chamber (1) and therefore over the active area of the support as long as the injection valve (13) is not switched to its initial position.

In the device according to FIG. 3, a manually actuated system (injection valve for example, pump, etc.) allows separation of the solution containing the secondary reagents from the system allowing the active area to be maintained under flow (14).

Thus, the device of the invention gives the possibility of keeping a reservoir with reagents which may be reused as often as required, within the limit of the stability of the reagent and of its possible gradual dilution during the recyclings: the excess reagent is no longer wasted.

Moreover, the advantage of this device lies in the fact that the analysis of the samples is simplified: the secondary reagent is injected from the reservoir with the sample prior to any analysis. Thus, after having injected the liquid of interest, it is sufficient to connect the circuit containing the secondary reagents (15) to the chamber (1) in order to optionally amplify the signal, and to do the opposite operation in order to put an end to the secondary reagents/active area interaction.

Alternatively, the device may have several distinct secondary circuits (15) each containing a solution with one (or more) secondary reagent(s).

Thus, in both of these recycling configurations, this device gives the possibility of promoting the secondary reagents/active area interaction. Indeed, it is quite possible to have the excess secondary reagents continuously circulate over the active area, in order to optimize the contact between the secondary reagents and the active area.

Therefore, this device gives the possibility of achieving actual savings in terms of consumptions of reagents, since a same batch of secondary reagents may be used several times for analyzing various samples on the one hand; and the interactions are promoted without any additional consumption of a new batch of reagents on the other hand.

However it should be noted that this recycling cannot be unlimited, since each cycle causes a loss of reagents either towards the biological garbage bin, or by dilution because of the existence of a buffer/reagents interface. On the other hand, a fragile biological reagent may have limited stability. Therefore, the number of recyclings of a given reagent has to be adapted to the analytical needs in terms of sensitivity and reproducibility.

This method may be adapted to any biological analysis applying at least one secondary reagent, whether this be relative to DNA analysis, immuno-analysis . . . .

III. Application of a Method According to the Invention.

III.1. Principle and Equipment.

The embodiment shown here refers to the case when the excess of reagents is recovered in a recycling route which may be isolated from the main fluidic circuit. One of the possible embodiments of the device for recycling is described in FIG. 3. For this embodiment, Rheodyne® injection valves are used.

The device according to the present invention is used within the scope of detection of toxins based on the use of a biochip with antibodies, coupled with an optical readout system, with which surface plasmon resonance may be achieved. These are gold nanoparticles which are recycled in this example. With these nanoparticles it is possible to amplify the signal in the case of a sample in which the concentration of analyte is too low.

The biosensor used consists of a prism on which is deposited a gold layer with a thickness of 50 nm. The gold layer is functionalized by means of grafting anti-toxin antibodies of interest via a polypyrrole film.

In order to ensure the reliability of this technique for detecting the toxin, [symbol] negative control [symbol] areas are made on the surface of the sensor. These areas consist of species which do not have any specific affinity for the toxin to be detected.

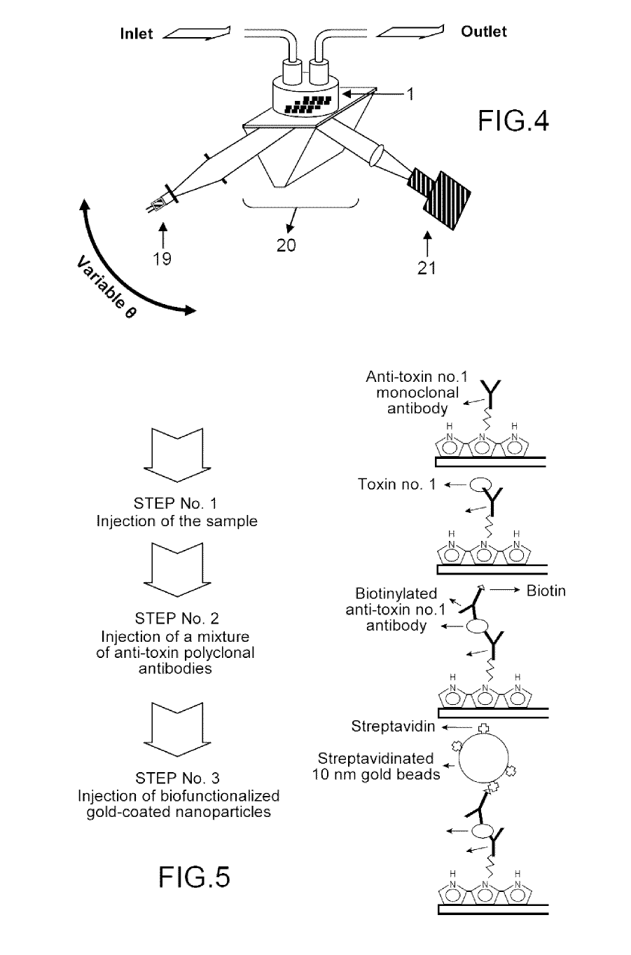

From a practical point of view, the apparatus used is like the one illustrated in FIG. 4. A light emitting diode (LED) (19) illuminates from beneath the golden glass prism which makes up the support (20) of the active area. This illumination is achieved under a fixed angle for which surface plasmon resonance (SPR) occurs. The reflected ray is then sensed by a CCD (Charge Coupled Device) camera (21) itself connected to a computer. Acquisition of reflectivity in real time is achieved by means of a software package marketed by Genoptics®, Genovision. The assembly of the fluidic system consists of PEEK tubes with an inner diameter of 0.8 mm. The experimental medium used is a saline phosphate buffer. In FIG. 4, the terms of [symbol] Inlet [symbol] and [symbol] Outlet [symbol] materialize the direction of flow of the experimental medium and the direction of injection of the samples possibly containing the toxin. The reference (1) corresponds to the chamber for which the base is formed by all or part of the surface of the support having active areas and [symbol] negative control [symbol] areas.

At set angle of incidence and wavelength, the biological interactions at the experimental medium/metal interface are detected by studying the change in the intensity of the reflected beam. More particularly, the adsorption of the target molecules at the surface of the biosensor causes a local change in reflectivity.

This assembly is placed in a dark chamber regulated to 25° C.

111.2. Method.

The sample containing a given toxin is first injected under constant flow over the biosensor. Under adequate flow rate and concentration conditions, the specific anti-toxin antibody/toxin interaction causes specific attachment of the toxin on the corresponding antibody pads present on the biosensor.

Next, the reflectivity signal is amplified for a first time by means of the injection of anti-toxin antibodies functionalized with biotin (fictitious increase in the mass of the analyte).

Finally, the last amplification step is carried out by the attachment of gold nanoparticles functionalized by streptavidin (FIG. 5).

Within the scope of the method according to the invention, the gold nanoparticles are the ones which are recycled in the case of detection of a toxin shown hereafter. These gold nanoparticles according to the definitions of the present invention form a secondary reagent indirectly bound to the analyte and directly detectable.

To do this, after having filled the closed circuit with a sample of gold nanoparticles, a constant flow is maintained in the reservoir thereby formed with gold nanoparticles. The biosensor is maintained under permanent flow of an experimental buffer equal to 50 μL/min.

The experiment takes place on the same biosensor, in two phases: first analysis of a blank sample, i.e. not containing any target toxin, and then analysis of a sample containing a small amount of target toxin.

For each analyzed sample, the procedure is identical. After having injected the sample via the 1^{st} injection valve, a new sample containing biotinylated target anti-toxin antibodies is injected via the same injection valve. Finally, the 2^{nd} injection valve is manually actuated thereby allowing the gold nanoparticles to be present in the active area circuit.

III.3. Results.

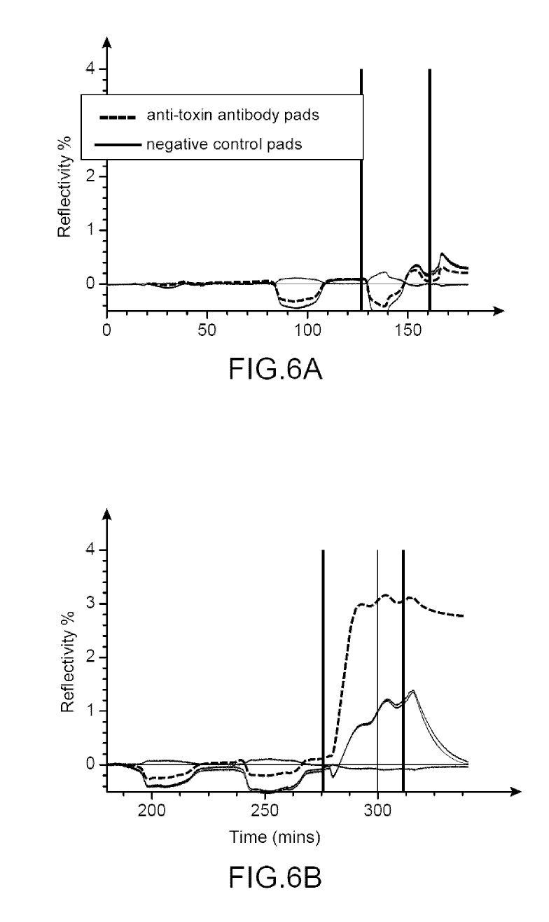

The results of both of these analyses are shown in FIG. 6. The upper graph (FIG. 6A) shows the kinetics of reflectivity during the gradual successive injections. The reflectivity level after injection of gold nanoparticles is similar to the initial reflectivity level and does not differ very much from one species to another. No adsorption of toxin is detected.

The graph of FIG. 6B shows the time-dependent change in reflectivity corresponding to the analysis of the sample containing the toxin to be detected. After the interaction between the active area and the gold nanoparticles and the return to an experimental buffer, a change in reflectivity is only observed on the area sensitive to the toxin. The observed variation of reflectivity is therefore actually specific to the presence of toxin in the analyzed sample.

Thus, the gold nanoparticles having circulated a first time over the active area, were able to be reused successfully in order to detect the presence of toxin in the second analyzed sample.

### REFERENCES

[1] Livache T, Maillart E, Lassalle N, et al. “Polypyrrole based DNA hybridation assays: study of label free detection processes versus fluorescence on microchips”. Journal of Pharmaceutical and Biomedical Analysis. 2003; 32(4-5): 687-696.
[2] Delehanty J B, Ligler F S. “A micro array immunoassay for simultaneous detection of proteins and bacteria.” Anal Chem. 2002; 74(21): 5681-7.
[3] U.S. Pat. No. 6,605,265 in the name of Atochem published on Aug. 12, 2003.
[4] International application WO 98/49187 in the name of Chiron Corporation published on Nov. 5, 1998.
[5] International application WO 02/051856 in the name of the CEA published on Jul. 4, 2002.
[6] International application WO 00/36145 in the name of the CEA published on Jun. 22, 2000.
[7] Tabeling P. “Introduction to Microfluidics” Oxford University Press. 2006.

1. A method for detecting and optionally quantifying an analyte possibly present in a liquid of interest, comprising the following steps:
(i) putting said liquid of interest in contact with the surface of a solid support comprising at least one active area on which at least one probe capable of binding said analyte is immobilized;
(ii) putting said surface in contact with a solution containing at least one either directly or indirectly detectable secondary reagent, capable of binding to the analyte, either directly or indirectly;
(iii) detecting and optionally quantifying said secondary reagent immobilized on said active area, either directly or indirectly;
wherein said method further comprises a step or recycling said solution so that it is prepared to be put back into contact with the surface at least once.

2. The method according to claim 1, further comprising
putting said surface into contact with the recycled solution and optionally
recycling said solution again.

3. The method according to claim 1, comprising the following steps:
a1) putting a 1st liquid into contact with the surface of a solid support comprising at least one active area on which at least one probe capable of binding said analyte is immobilized;
b1) putting said surface in contact with at least one either directly or indirectly detectable secondary reagent, capable of binding to the analyte either directly or indirectly;
c1) recycling said secondary reagent which has not been immobilized on said active area by direct or indirect binding with the analyte;
d1) detecting and optionally quantifying said secondary reagent immobilized on said active area, either directly or indirectly;
a2) putting said liquid of interest in contact with said surface;
b2) putting said surface in contact with at least the secondary reagent, recycled during said step (c1);
d2) detecting and optionally quantifying said secondary reagent immobilized on said active area, either directly or indirectly.

4. The method according to claim 1, comprising the following steps:
a1′) putting a 1st liquid into contact with the surface of a solid support comprising at least one active area on which at least one probe capable of binding said analyte is immobilized;
b1′) putting said surface in contact with at least one either directly or indirectly detectable secondary reagent, capable of binding to the analyte, either directly or indirectly;
c1′) recycling said secondary reagent which has not immobilized on the active area by direct or indirect binding with the analyte;
d1′) detecting and optionally quantifying said secondary reagent immobilized on the active area, either directly or indirectly;
a2′) putting said liquid of interest in contact with said surface;
b2′) putting said surface into contact with at least the secondary reagent, recycled during said step (c1′);
c2′) recycling said secondary reagent which has not immobilized on the active area by direct or indirect binding with the analyte so as to repeat at least once a step (b2′) and optionally step (c2′);
d2′) detecting and optionally quantifying, said secondary reagent immobilized on the active area either directly or indirectly.

5. The method according to claim 1, wherein the liquid of interest is selected from the group consisting of a biological fluid, a plant fluid, a sampling in a culture medium or in a biological culture reactor, a liquid obtained from one or more animal or plant cell(s), a liquid obtained from animal or plant tissue, a sampling in a food matrix, a sampling in a chemical reactor, tap water, river water, sea water, water from air-cooled towers, an air sample, a sample from a liquid or gas industrial effluent, an earth sample and a mixture thereof.

6. The method according to claim 1, wherein said analyte to be detected and optionally quantified is selected from the group consisting of a molecule of biological interest, a molecule of pharmacological interest, a toxin, a carbohydrate, a peptide, an antigen, an epitope, a protein, a glycoprotein, an enzyme, an enzymatic substrate, a nuclear or membrane receptor, an agonist or antagonist of a nuclear or membrane receptor, a hormone, a polyclonal or monoclonal antibody, an antibody fragment, a nucleotide molecule, an eukaryotic cell and a prokaryotic cell.

7. The method according to claim 1, wherein said probe is selected from the group consisting of a carbohydrate, a peptide, an antigen, an epitope, a protein, a glycoprotein, an enzyme, an enzymatic substrate, a membrane or nuclear receptor, an agonist or antagonist of a membrane or nuclear receptor, a toxin, a polyclonal or monoclonal antibody, an antibody fragment, a nucleotide molecule, a peptide nucleic acid and an aptamer.

8. The method according to claim 1, wherein the direct or indirect detection and the optional quantification of the secondary reagent apply techniques without any marker.

9. The method according to claim 1, wherein the direct or indirect detection and the optional quantification of the secondary reagent apply a marker selected from the group consisting of a color particle, a fluorophore or a fluorescent marker, a phosphorescent marker, a chemiluminescent marker, a chemical molecule, an electrochemically active molecule, a biologically active molecule capable of producing a detectable signal when incubated with the enzyme or the adequate chromogenic substrate and a radioactive marker.

10. A device comprising:
a chamber comprising a solid support, a surface of which comprises at least one active area, which may be functionalized with at least one probe capable of binding an analyte;
a 1st fluidic system configured to circulate a liquid of interest, which may contain said analyte over said surface;
a 2nd fluidic system comprising at least one reservoir containing a solution comprising at least one secondary reagent, said fluidic system being configured to circulate a solution comprising at least one secondary reagent at least twice in the same direction over said surface.

11. A device comprising:
a chamber comprising a solid support, a surface of which comprises at least one active area which may be functionalized with at least one probe capable of binding an analyte;
a 1st fluidic system configured to circulate a liquid of interest which may contain said analyte on said surface;
a 2nd fluidic system configured to circulate at least one solution comprising at least one secondary reagent at least once in one direction and at least one other time in the opposite direction over said surface.

12. The device according to claim 11, wherein said 2nd fluidic system comprises one or more upstream reservoir(s) containing the solution(s) comprising at least one secondary reagent, and optionally one or more reservoir(s) positioned downstream containing or which may contain the solution(s) comprising at least one secondary reagent.

13. The method of claim 8, wherein said detection and optional quantification comprise techniques using surface plasmon resonance (SPR) or quartz scales.
